(19) United States
(12) Patent Application Publication
(10) Pub. No.: US 2016/0367165 A1
(43) Pub. Date: Dec. 22, 2016

(54) NON-INVASIVE METHOD FOR ASSESSING AND MONITORING BRAIN INJURIES

(71) Applicant: THE REGENTS OF THE UNIVERSITY OF CALIFORNIA, Oakland, CA (US)

(72) Inventor: Syed Khizer Rahim KHADERI, Davis, CA (US)

(21) Appl. No.: 14/902,320

(22) PCT Filed: Jul. 2, 2014
(86) PCT No.: PCT/US2014/045321

§ 371 Date: Dec. 30, 2015

Related U.S. Application Data
(60) Provisional application No. 61/842,939, filed on Jul. 3, 2013.

(51) Int. Cl. A61B 5/0484; A61B 5/00; A61B 5/04; A61B 3/113

(52) U.S. Cl. CPC A61B 5/04842; A61B 3/113; A61B 5/4064; A61B 5/04012; A61B 5/6814

(57) ABSTRACT

A method of assessing a brain injury of a patient that comprises performing a brain wave test, performing a pupillary response test, and performing an eye tracking test, then generating a brain injury score based on the results of the brain wave test, the pupillary response test, and the eye tracking test. In some examples, the brain injury score is determined by comparing the test results with a normative database of reference results.

### CROSS REFERENCE TO RELATED APPLICATIONS

This application claims priority to U.S. Provisional Application No. 61/842,939, filed Jul. 3, 2013. The content of the above-referenced application is incorporated herein by reference for all purposes.

### BACKGROUND

1. Field

The present disclosure relates generally to assessment of brain wave function, and more specifically to combining the results of eye tracking tests, pupillary response tests, and brain wave tests to assess and monitor brain injuries.

2. Description of Related Art

Traumatic brain injuries (TBIs) are a significant health concern with both short- and long-term ramifications. TBIs are more commonly seen in athletes and soldiers due to their relatively high risk of head trauma, but such injuries may occur in many settings.

Accurately assessing the severity of potential traumatic brain injuries in the field is difficult. First responders are often limited to a basic physical assessment of vital signs coupled with a qualitative assessment based on subjective information provided by the patient (if conscious) or by observers.

Such evaluations are not always reliable, and provide only a rough assessment of the severity of a TBI. Furthermore, it may not be possible to obtain some or all of the qualitative information needed, particularly if the patient is unable to recall the accident and there were no observers. Therefore, a standardized, quantitative method for assessing traumatic brain injuries in the field is needed.

### BRIEF SUMMARY

A method of assessing a brain injury of a patient, comprising performing a brain wave test, performing a pupillary response test, and performing an eye tracking test, then generating a brain injury score based on the results of the brain wave test, the pupillary response test, and the eye tracking test. In some examples, the results of the first test are used to calculate a first score, the results of the second test are used to calculate a second score, and the results of the third test are used to calculate a third score, and the first score, the second score, and the third score are combined to generate a total score. In some examples, the first score, the second score, and the third score are normalized before being combined to generate a total score. In some examples, the total score is compared to a normative database of reference scores to generate the brain injury score.

### DESCRIPTION OF THE FIGURES

FIG. 1 depicts an exemplary method for generating a brain injury score.

FIG. 2 depicts an exemplary method for conducting eye-tracking tests.

FIG. 3 depicts an exemplary method of conducting pupillary tests.

FIG. 4 depicts an exemplary method of conducting brain wave tests.

FIG. 5 depicts an exemplary method of combining test results to generate a brain injury score.

FIG. 6A depicts an exemplary apparatus for use in performing brain wave tests.

FIG. 6B depicts an exemplary apparatus for use in performing eye-tracking tests and pupillary response tests.

FIG. 7 depicts an exemplary system for use in assessing and monitoring traumatic brain injuries.

FIG. 8 depicts exemplary visual stimulation for a pro-saccade test.

FIGS. 9A-B depict exemplary visual stimulation for an anti-saccade test.

FIG. 10 depicts exemplary visual stimulation for a smooth pursuit test.

FIG. 11 depicts exemplary visual stimulation for a fade in, fade out smooth pursuit test.

FIG. 12 depicts exemplary visual stimulation for a pupillary response test.

FIG. 13 depicts exemplary variables for use in a system for assessing and monitoring traumatic brain injuries.

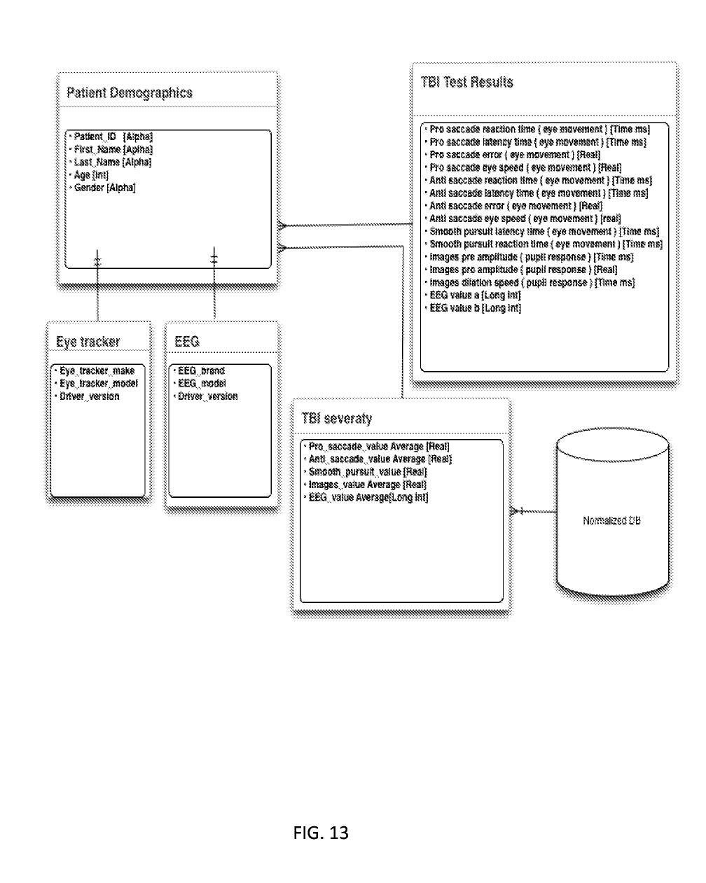

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

This disclosure describes processes for assessing and monitoring traumatic brain injuries by performing a series of eye tracking, pupillary, and brain wave tests using a set of standardized visual stimuli, and using the test results to generate a composite brain injury score based on comparisons of the results to a normative reference database. In contrast to traditional methods for assessing and monitoring traumatic brain injuries, the currently disclosed methods enable non-invasive, standardized, quantitative TBI assessments that may be performed in the field.

3. Method for Assessing a Brain Injury

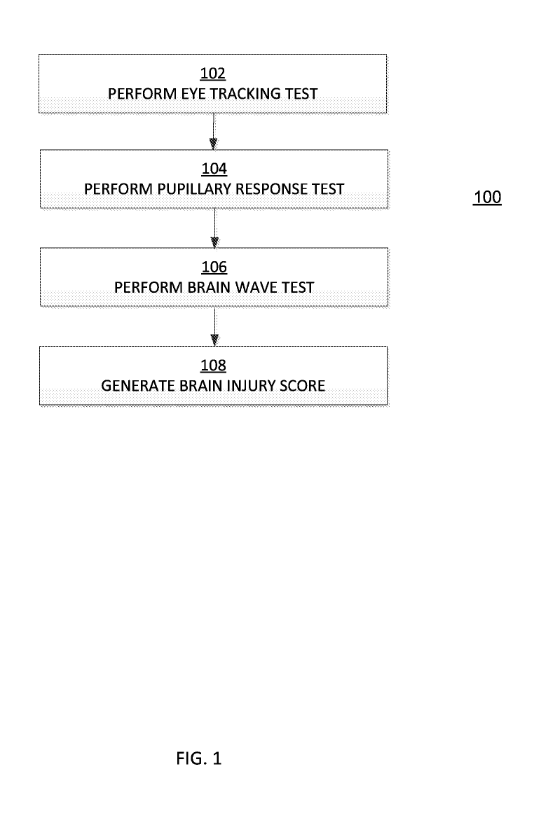

FIG. 1 depicts an exemplary method 100 for assessing a brain injury.

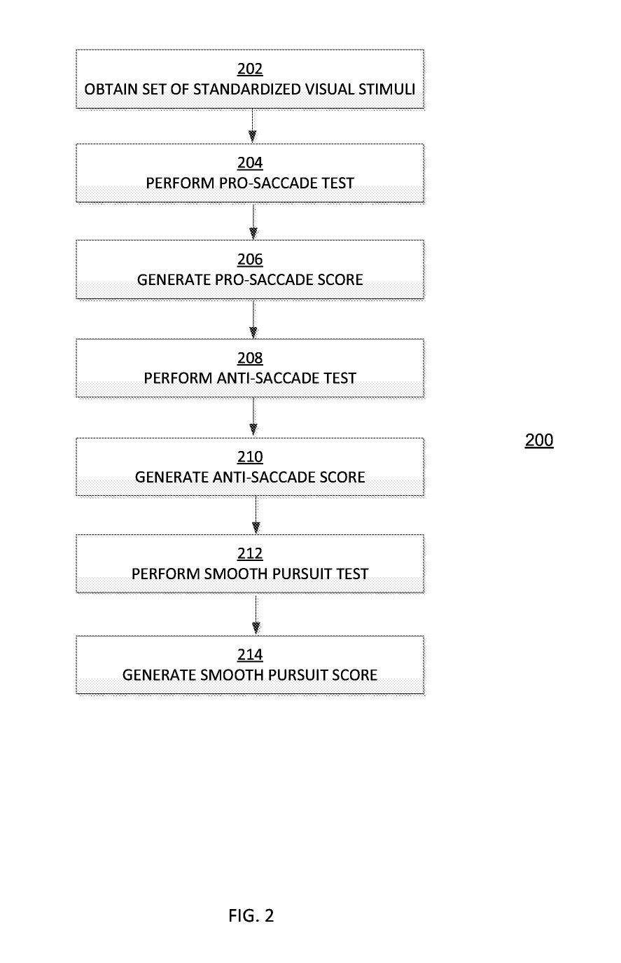

In block 102, an eye tracking test is performed. In some embodiments, the eye tracking test may be performed as depicted by FIG. 2 and described for exemplary process 200.

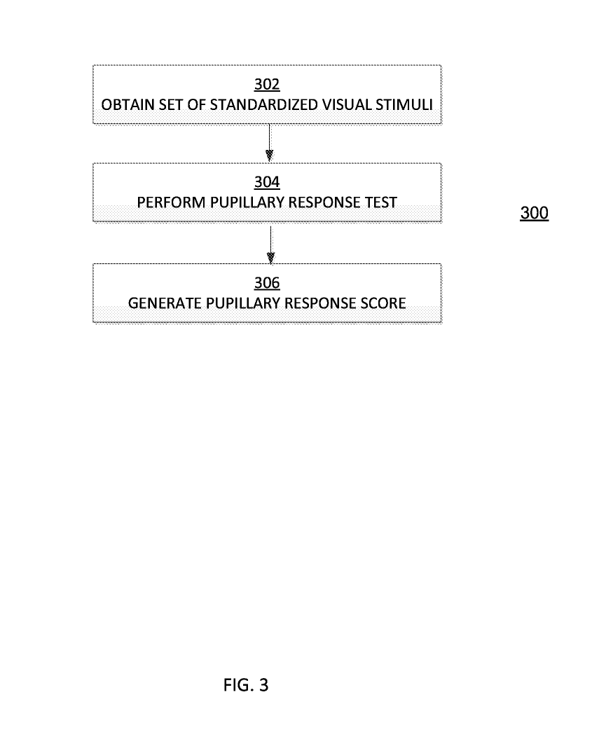

In block 104, a pupillary response test is performed. In some embodiments, the pupillary response test may be performed as depicted in FIG. 3 and described for exemplary process 300.

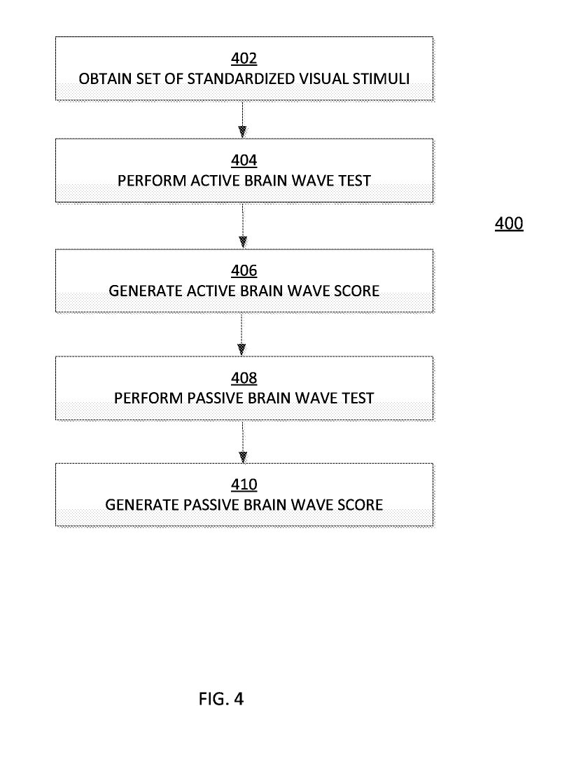

In block 106, a brain wave test is performed. In some embodiments, the brain wave test may be performed as depicted in FIG. 4 and described for exemplary process 400.

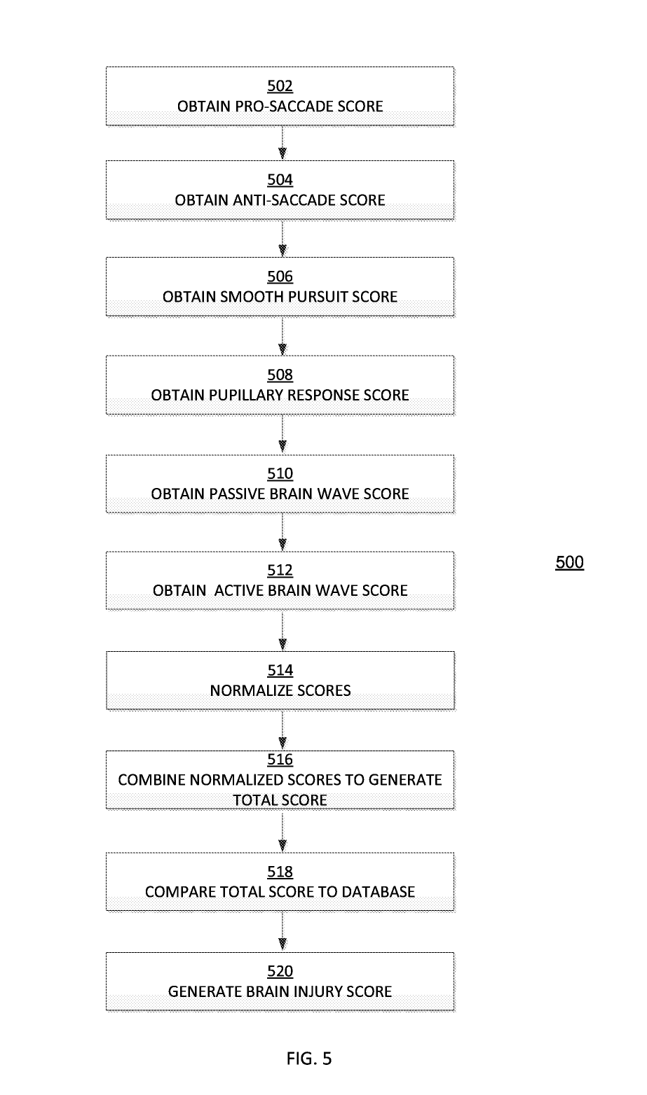

In block 108, a brain injury score is generated. In some embodiments, the brain injury score may be generated as depicted in FIG. 5 and described for exemplary process 500.

The overall method depicted in FIG. 1 is described in more detail with respect to FIGS. 2-5, below.
a. Perform Eye Tracking Test

FIG. 2 depicts an exemplary process 200 for assessing eye tracking.

In block 202, a set of standardized visual stimuli is obtained. In a preferred embodiment, this set of standardized visual stimuli includes stimuli designed to elicit responses appropriate for assessing eye-tracking performance. The set of visual stimuli may be obtained from computer memory, from a CD or thumb drive, or from a remote server, for example.

In block 204, a pro-saccade eye tracking test is performed. The pro-saccade test measures the amount of time required for a patient to shift his or her gaze from a stationary object towards a flashed target. The pro-saccade eye tracking test may be conducted as described in The Antisaccade: A Review of Basic Research and Clinical Studies, by S. Everling and B. Fischer, Neuropsychologia Volume 36, Issue 9, 1 Sep. 1998, pages 885-899 (“Everling”), for example.

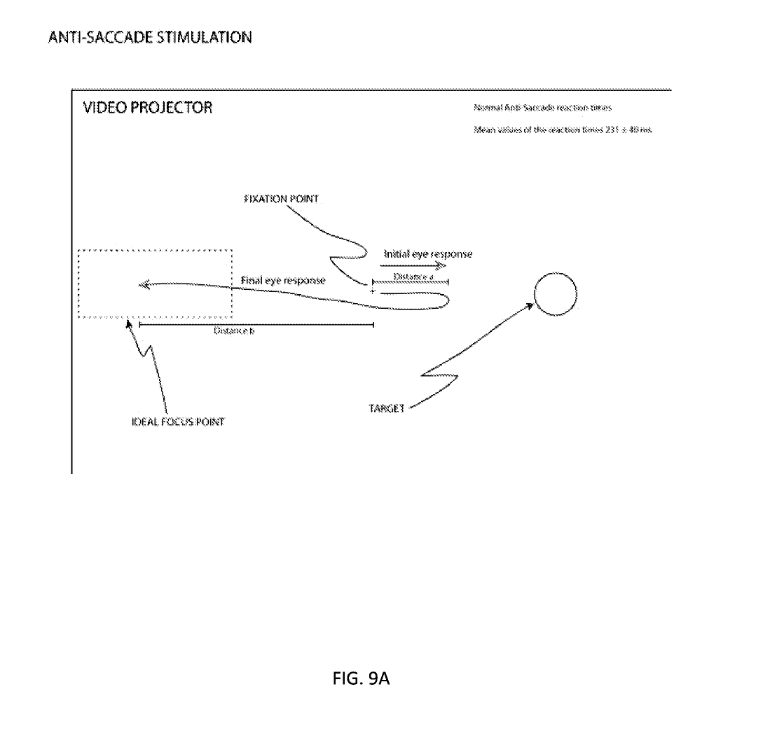

The pro-saccade test may be performed while presenting the patient with the standardized set of visual stimuli obtained in block 202. In some embodiments, the pro-saccade test may be conducted multiple times with the same or different stimuli to obtain an average result. FIG. 8 depicts exemplary visual stimulation for a pro-saccade test.

The results of the pro-saccade test may comprise, for example, the pro-saccade reaction time. The pro-saccade reaction time is the latency of initiation of a voluntary saccade, with normal values falling between roughly 200-250 ms. Pro-saccade reaction times may be further sub-grouped into:

Express Pro-Saccades: 80-134 ms

Fast regular: 135-175 ms

Slow regular: 180-399 ms

Late: (400-699 ms)

In block 206, a pro-saccade score is generated. The pro-saccade score may be generated using the results of the pro-saccade test performed in block 204. The pro-saccade score may be generated by comparing the results of the pro-saccade test performed in block 204 with a database of normative reference results for pro-saccade tests conducted using the same set of visual stimuli as obtained in block 202. In some embodiments, the pro-saccade score may be represented as one or more values of the form +/−n, where n is the difference between a pro-saccade result and a normative pro-saccade result from a reference database.

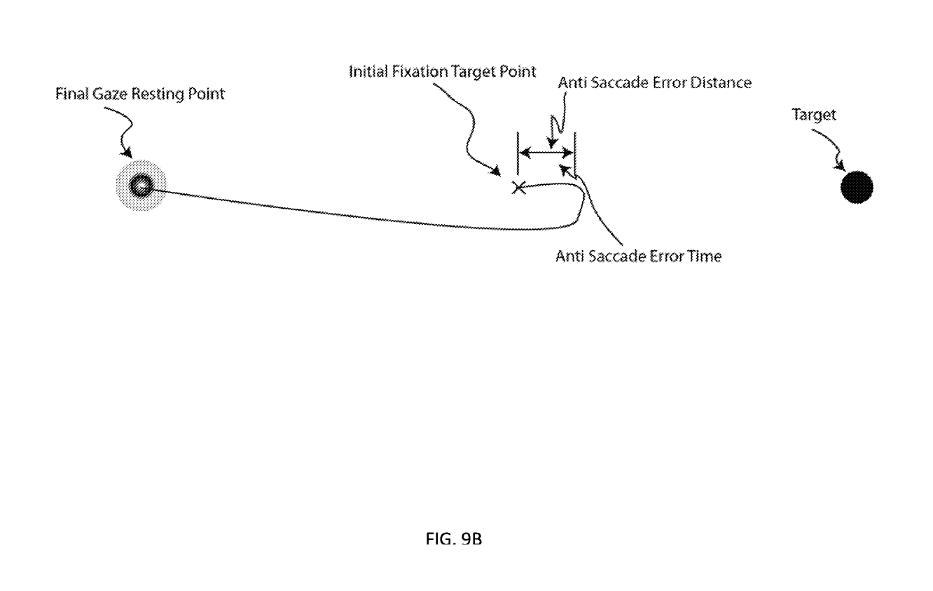

In block 208, an anti-saccade eye tracking test is performed. The anti-saccade test measures the amount of time required for a patient to shift his or her gaze from a stationary object away from a flashed target, towards a desired focus point. The anti-saccade eye tracking test can be conducted as described in Everling, for example. In some examples, the anti-saccade test may also measure an error time and/or error distance; that is, the amount of time or distance in which the eye moves in the wrong direction (towards the flashed target). The anti-saccade test may be performed using the standardized set of visual stimuli obtained in block 202. FIGS. 9A-B depict exemplary visual stimulation for an anti-saccade test.

The results of the anti-saccade test may comprise, for example, mean reaction times as described above for the pro-saccade test, with typical mean reaction times falling into the range of roughly 190 to 270 ms. Other results may include initial direction of eye motion, final eye resting position, time to final resting position, initial fovea distance (i.e., how far the fovea moves in the direction of the flashed target), final fovea resting position, and final fovea distance (i.e., how far the fovea moves in the direction of the desired focus point).

In block 210, an anti-saccade score is generated. The anti-saccade score may be generated using the results of the anti-saccade test performed in block 208. The anti-saccade score may be generated by comparing the results of the anti-saccade test performed in block 208 with a database of normative reference results for anti-saccade tests conducted using the same set of standardized visual stimuli as obtained in block 202. In some embodiments, the anti-saccade score may be represented as one or more values of the form +/−n, where n is the difference between an anti-saccade test result and a normative anti-saccade result from a reference database.

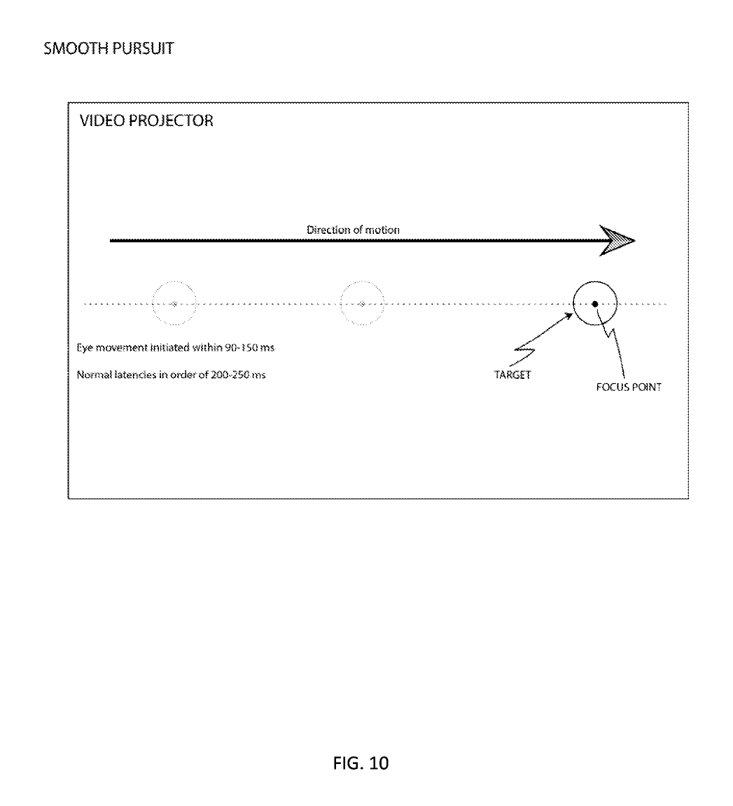

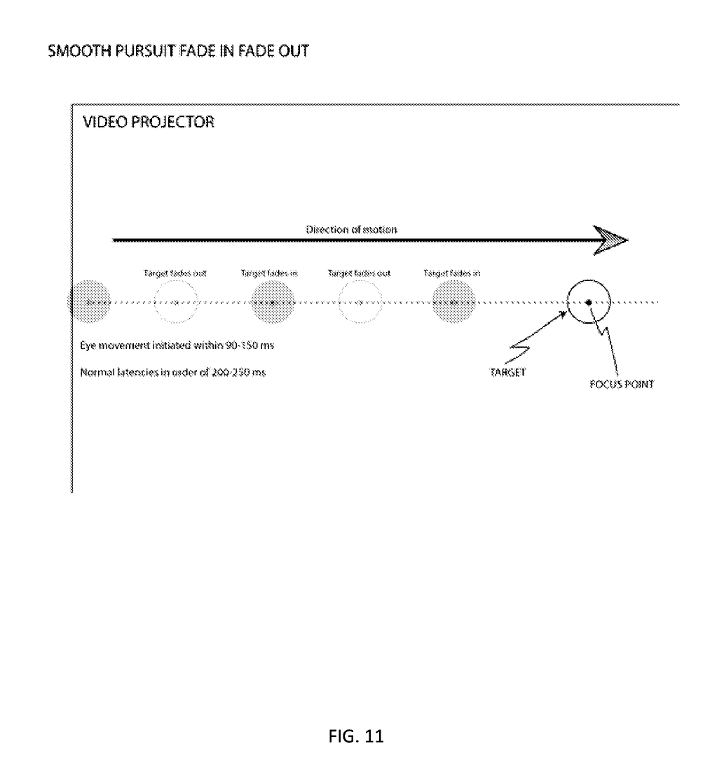

In block 212, a smooth pursuit test is performed. The smooth pursuit test evaluates a patient's ability to smoothly track moving visual stimuli. The smooth pursuit test can be conducted by asking the patient to visually follow a target as it moves across the screen. The smooth pursuit test may be performed using the standardized set of visual stimuli obtained in block 202, for example, and may be conducted multiple times with the same or different stimuli to obtain an average result. In some embodiments, the smooth pursuit test may include tests based on the use of fade-in, fade-out visual stimuli, in which the target fades in and fades out as the patient is tracking the target. FIG. 10 depicts exemplary visual stimulation for a smooth pursuit test. FIG. 11 depicts exemplary visual stimulation for a fade-in, fade-out smooth pursuit test.

Data gathered during the smooth pursuit test may comprise, for example, an initial response latency and a number of samples that capture the fovea position along the direction of motion during target tracking. Each sampled fovea position may be compared to the position of the center of the target at the same time to generate an error value for each sample.

In block 214, a smooth pursuit score is generated. The smooth pursuit score may be generated using the results of the smooth pursuit test performed in block 212, including the initial latency, the error values, and elapsed time to final position. The average range for initiation of pursuit is 90-150 ms; typical elapsed times to final position are on the order of 200-250 ms. The smooth pursuit score may be generated by comparing the results of the smooth pursuit test performed in block 212 with a database of normative reference results for smooth pursuit tests conducted using the same set of standardized visual stimuli as obtained in block 202. In some embodiments, the smooth pursuit score may be represented as one or more values of the form +/−n, where n is the difference between a smooth pursuit test result and a normative smooth pursuit result from a reference database.
b. Perform Pupillary Response Test

FIG. 3 depicts an exemplary process 300 for assessing pupillary response.

In block 302, a set of standardized visual stimuli is obtained. In a preferred embodiment, this set of standardized visual stimuli includes stimuli that are designed to elicit responses appropriate for assessing pupillary response, such as the stimuli described below.

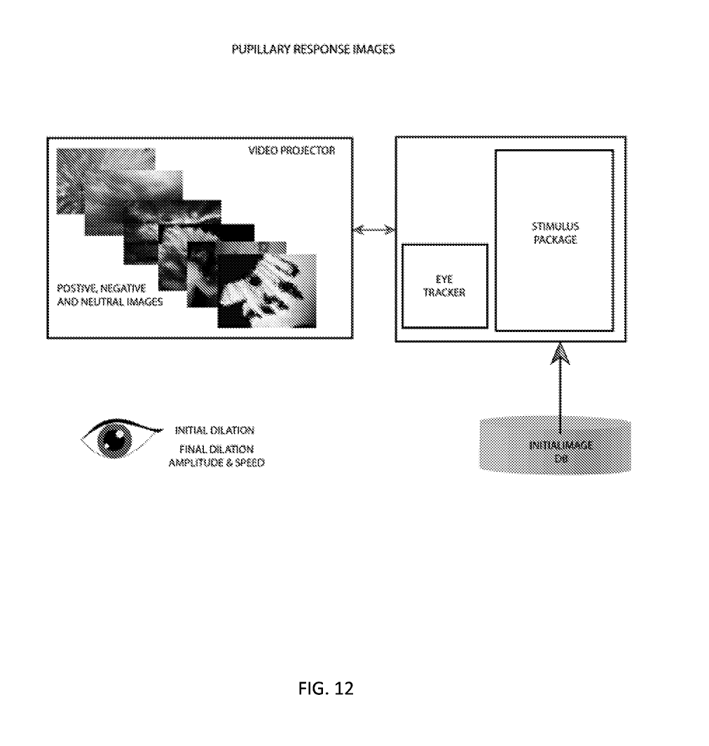

In a hospital setting, pupillary response is often assessed by shining a bright light into the patient's eye and assessing the response. In field settings, where lighting is difficult to control, pupillary response may be assessed using a standardized set of photographs, such as the International Affective Picture System (IAPS) standards. These photographs have been determined to elicit predictable arousal patterns, including pupil dilation, and may serve as the set of standardized visual stimuli for exemplary process 300. The set of visual stimuli may be obtained from computer memory, from a CD or thumb drive, or from a remote server, for example. FIG. 12 depicts exemplary visual stimulation for a pupillary response test.

In block 304, a pupillary response test is performed. The pupillary response test may be conducted by taking an initial reading of the patient's pupil diameter, pupil height, and/or pupil width, then presenting the patient with visual stimuli to elicit a pupillary response. The change in pupil dilation (e.g., the change in diameter, height, width, and/or an area calculated based on some or all of these measurements) and the time required to dilate are measured.

The pupillary response test may be performed using a variety of stimuli, such as changes to lighting conditions (including shining a light in the patient's eyes), or presentation of photographs, videos, or other types of visual data. In some embodiments, the pupillary response test is conducted while presenting the standardized set of visual stimuli obtained in block 302. In some embodiments, the pupillary test may be conducted multiple times with the same or different stimuli to obtain an average result.

The results of the pupillary response test may include, for example, a set of dilation (mydriasis) results and a set of contraction (miosis) results, where each set may include amplitude, velocity (speed of dilation/constriction), pupil diameter, pupil height, pupil width, and delay to onset of response.

In block 306, a pupillary response score is generated. The pupillary response score may be generated using the results of the pupillary response test performed in block 304. In some embodiments, the pupillary response score may be generated by comparing the results of the pupillary response test performed in block 304 with a database of normative reference results for pupillary response tests conducted using the same set of standardized visual stimuli as obtained in block 302. In some embodiments, the pupillary response score may be represented as one or more values of the form +/−n, where n is the difference between a pupillary response result and a normative pupillary response result from a reference database.
c. Perform Brain Wave Test

FIG. 4 depicts an exemplary process 400 for assessing brain wave activity.

In block 402, a set of standardized visual stimuli is obtained. In a preferred embodiment, this set of standardized visual stimuli includes a subset of visual stimuli designed to elicit responses appropriate for assessing active brain wave activity.

In block 404, an active brain wave test is performed. The active brain wave test may be conducted using EEG (electroencephalography) equipment and following methods known in the art. The active brain wave test may be performed while the patient is presented with a variety of visual stimuli. In some embodiments, the active brain wave test is conducted while presenting the standardized set of visual stimuli obtained in block 402. In some embodiments, the active brain wave test may be conducted multiple times, using the same or different visual stimuli, to obtain an average result. The results of the active brain wave test may comprise, for example, temporal and spatial measurements of alpha waves, beta waves, delta waves, and theta waves. In some embodiments, the results of the active brain wave test may comprise a ratio of two types of brain waves; for example, the results may include a ratio of alpha/theta waves.

In block 406, an active brain wave function score is generated. The active brain wave score may be generated using the results of the active brain wave test performed in block 404. In some embodiments, the active brain wave score may be generated by comparing the results of the active brain wave test performed in block 404 with a database of normative reference results for brain wave tests conducted using the same set of standardized visual stimuli as obtained in block 402. In some embodiments, the active brain wave score may be represented as one or more values of the form +/−n, where n is the difference between an active brain wave test result and a normative active brain wave result from a reference database.

In block 408, a passive brain wave test is performed. The passive brain wave test may be conducted using EEG (electroencephalography) equipment to record brain wave data while the patient has closed eyes; i.e., in the absence of visual stimuli. The results of the passive wave brain wave test may comprise, for example, temporal and spatial measurements of alpha waves, beta waves, delta waves, and theta waves, for example. In some embodiments, the results of the passive brain wave test may comprise a ratio of two types of brain waves; for example, the results may include a ratio of alpha/theta waves. In some embodiments, the passive brain wave test may be conducted multiple times to obtain an average result.

In block 410, a passive brain wave score is generated. The passive brain wave score may be generated using the results of the passive brain wave test performed in block 408. In some embodiments, the passive brain wave score may be generated by comparing the results of the passive brain wave test performed in block 408 with a database of normative reference results for passive brain wave tests. The score may be generated using some or all of the results produced by the tests conducted in block 408. In some embodiments, the passive brain wave score may be represented as one or more values of the form +/−n, where n is the difference between a passive brain wave test result and a normative passive brain wave result from a reference database.
d. Generating a Brain Injury Score

FIG. 5 depicts an exemplary process 500 for generating a brain injury score.

In block 502, a pro-saccade score is obtained. In some embodiments, the pro-saccade score may be generated as described in exemplary process 200.

In block 504, an anti-saccade score is obtained. In some embodiments, the anti-saccade score may be generated as described in exemplary process 200.

In block 506, a smooth pursuit score is obtained. In some embodiments, the smooth pursuit score may be generated as described in exemplary process 200.

In block 508, a pupillary response score is obtained. In some embodiments, the pupillary response score may be generated as described in exemplary process 300.

In block 510, a passive brain wave score is obtained. In some embodiments, the passive brain wave score may be generated as described in exemplary process 400. In some embodiments, the passive brain wave score may be generated based on a ratio of two types of brain waves; for example, the passive brain wave score may be generated based on a ratio of alpha waves to theta waves.

In block 512, an active brain wave score is obtained. In some embodiments, the active brain wave score may be generated as described in exemplary process 400. In some embodiments, the active brain wave score may be generated based on a ratio of two types of brain waves; for example, the active brain wave score may be generated based on a ratio of alpha waves to theta waves.

In block 514, the pro-saccade score, anti-saccade score, smooth pursuit score, pupillary response score, passive brain wave score, and active brain wave score are normalized. In some embodiments, the n values may be normalized such that scores having inherently larger numerical variations of n will not dominate the overall results. Such normalization may be performed according to normalization methods known in the art.

In block 516, the normalized pro-saccade score, anti-saccade score, smooth pursuit score, pupillary response score, passive brain wave score, and active brain wave score generated in block 514 are used, in combination, to generate a total score. In some embodiments, the normalized scores may be weighted before being used to generate the total score.

In block 518, the total score computed in block 516 is compared to a normative database of total scores to generate a brain injury score. In some embodiments, the brain injury score is generated using typical analytic methods such as regression analysis, for example.

4. Creating a Normative Database

The normative database used as a reference for computing the test scores and brain injury scores may be created by performing the series of tests described in FIGS. 2-4 on a population of healthy individuals. In some embodiments, normative data may be categorized by age, gender, or other variables to enable more accurate comparisons between test data and reference data.

5. Apparatus and System

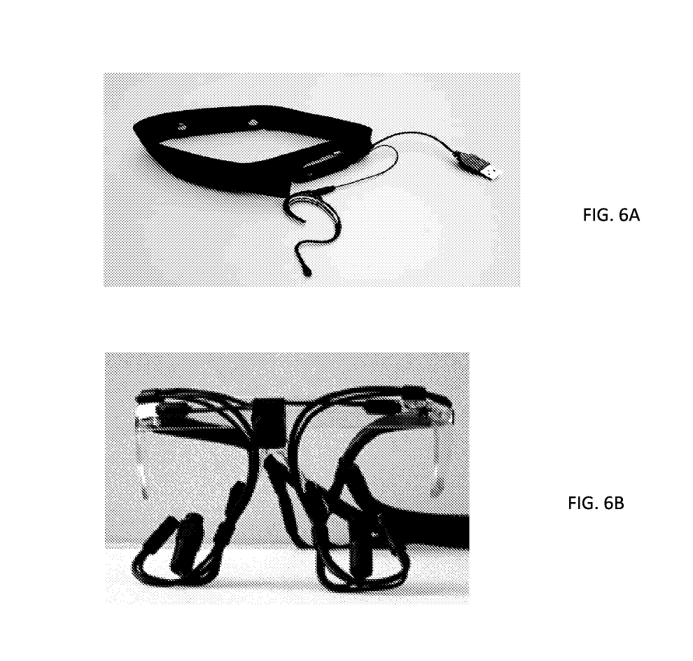

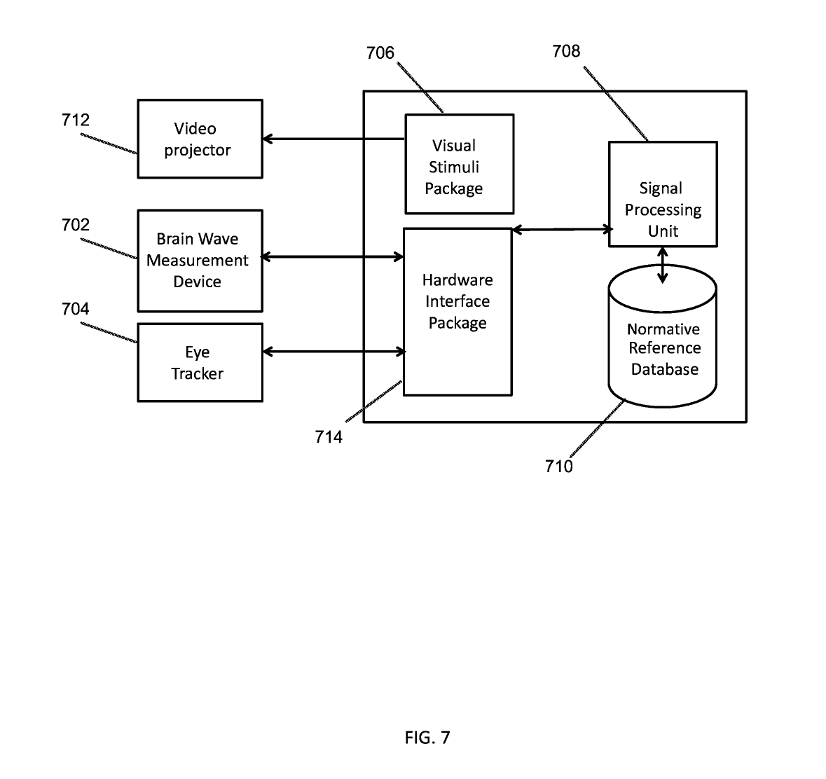

Examples of portable apparatus that may be used to perform the eye tracking, pupillary, and brain wave tests described above are shown in FIGS. 6A-B. FIG. 6A depicts a band with electrodes that may be worn on a patient's head and connected to a computer or other processing or memory device to perform EEG tests. This exemplary apparatus can be used to capture brain wave data while the patient is resting with eyes closed or while the patient is viewing visual stimuli, which may be presented on a laptop or projected onto a viewing screen, for example.

FIG. 6B depicts eye-tracking apparatus that may be worn in a manner similar to eyeglasses. This exemplary apparatus includes several cameras that can be used to capture eye tracking and pupillary response data while the patient is viewing visual stimuli, which may be presented on a laptop or projected onto a viewing screen, for example.

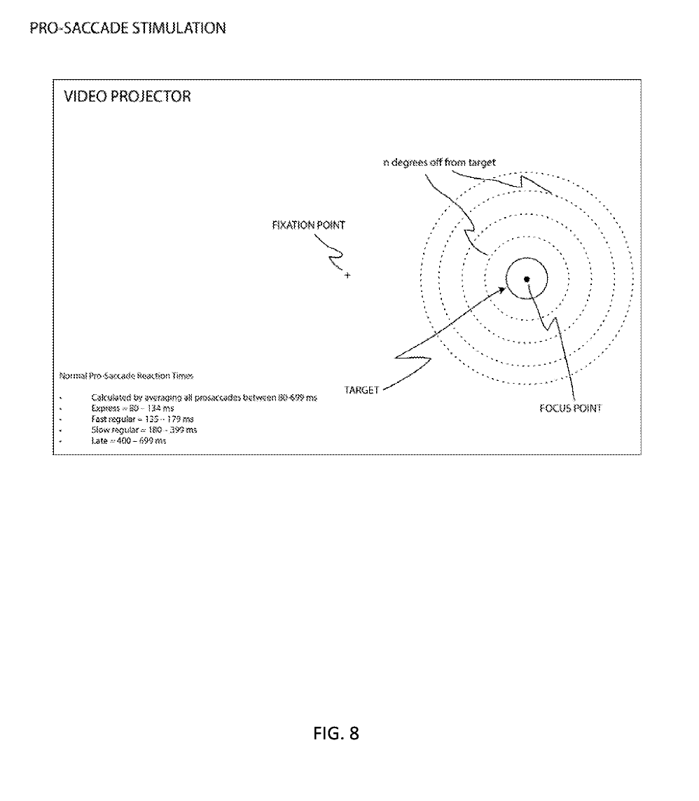

FIG. 7 depicts an exemplary system for assessing and monitoring traumatic brain injuries.

The brain wave measurement device 702 may be a device such as depicted in FIG. 6A, for example, and the eye tracker 704 may be a device such as depicted in FIG. 6B. The visual stimuli package 706 may be stored on a local computer's RAM or ROM, on a portable storage medium such as a CD or a thumb drive, or on a remote server, where it may be accessed by streaming or downloading the data. During testing, the visual stimuli package 706 may be displayed on a video projector 712, for example, or on other suitable display devices.

The hardware interface package 714 is designed to enable the system to accommodate outputs from multiple types of brain wave and eye tracking devices. The hardware interface package 714 receives data from the device for recording brain waves and from the eye tracking device and processes the data to convert it to an appropriate format for transmission to the signal processing unit 708. The hardware interface package 714 may also support bi-directional communications between the signal processing unit 708 and the eye tracker 704 if needed. Formatted data received from the hardware interface package 714 may be processed using a variety of signal processing algorithms in the signal processing unit 708 before being stored in the normative reference database or compared to existing data in the normative reference database 710. Such processing may be used to evaluate and compare the amplitudes of different brain waves, for example, to perform filtering, correlation, or other signal processing algorithms, or to otherwise assist in generating the test scores for the series of tests depicted in FIG. 1. The signal processing unit 708 may be implemented in hardware, software, or a combination of the two.

Although the invention has been described in considerable detail with reference to certain embodiments thereof, other embodiments are possible, as will be understood to those skilled in the art. Various exemplary embodiments are described herein. Reference is made to these examples in a non-limiting sense. They are provided to illustrate more broadly applicable aspects of the disclosed technology. Various changes may be made and equivalents may be substituted without departing from the true spirit and scope of the various embodiments. In addition, many modifications may be made to adapt to a particular situation, material, composition of matter, process, process act(s) or step(s) to the objective(s), spirit or scope of the various embodiments. Further, as will be appreciated by those with skill in the art, each of the individual variations described and illustrated herein has discrete components and features which may be readily separated from or combined with the features of any of the other embodiments without departing from the scope or spirit of the various embodiments.

What is claimed is:

1. A method of assessing a brain injury of a patient, the method comprising:
performing a brain wave test;
performing a pupillary response test;
performing an eye tracking test; and
generating a brain injury score based on the results of the brain wave test, the pupillary response test, and the eye tracking test.

2. The method of claim 1, further comprising:
generating a first score based on a comparison of the results of the brain wave test to a normative database of reference results;
generating a second score based on a comparison of the results of the pupillary response test to the normative database of reference results;
generating a third score based on a comparison of the results of the eye tracking test to the normative database of reference results; and
generating a brain injury score based on the first score, the second score, and the third score.

3. The method of claim 2, wherein the first score, the second score, and the third score are each normalized before generating the brain injury score.

4. The method of claim 1, wherein the pupillary response test is performed before the brain wave test.

5. The method of claim 1, wherein the eye tracking test is one or more tests selected from the group consisting of a pro-saccade test, an anti-saccade test, and a smooth pursuit test.

6. The method of claim 1, wherein the brain wave test is one or more tests selected from the group consisting of an active brain wave test and a passive brain wave test.

7. The method of claim 1, wherein the results of the brain wave test comprise a ratio of two types of brain waves.

8. The method of claim 7, wherein the ratio is a ratio of alpha waves to theta waves.

9. The method of claim 1, wherein the results of the eye tracking test comprise an error distance.

10. A method of assessing a brain injury of a patient, the method comprising:
performing a first test, the first test selected from the group consisting of a brain wave test, a pupillary response test, and an eye tracking test;
performing a second test, the second test selected from the group consisting of a brain wave test, a pupillary response test, and an eye tracking test; wherein the first test and the second test are different tests; and
generating a brain injury score based on the results of the first test and the second test.

11. A system for processing brain injury data for a patient, the system comprising:
a normative reference database that stores brain wave data, pupillary response data, eye tracking data, and total score data, wherein the brain wave data, pupillary response data, and eye tracking data were captured from one or more individuals while the one or more individuals were viewing a set of visual stimuli, and wherein the total score data is based on total scores generated for the one or more individuals based on the brain wave data, pupillary response data, and eye tracking data; and
a processor configured to:
receive pupillary response test results and eye tracking test results captured from the patient while the patient was viewing the set of visual stimuli;
receive brain wave test results;
generate a first score based on a comparison of the brain wave test results to the brain wave data stored in the normative database of reference results;
generate a second score based on a comparison of the pupillary response test results to the pupillary data stored in the normative database of reference results;
generate a third score based on a comparison of eye tracking test results to the eye tracking data stored in the normative database of reference results;
calculate a total score based on a combination of the first score, the second score, and the third score; and
generate a brain injury score based on a comparison of the total score to the total score data stored in the normative database of reference results.

12. The system of claim 11, further comprising:
the set of visual stimuli;
an eye tracking device;
a device for recording brain waves; and
an interface that receives data from the eye tracking device and the device for recording brain waves, formats the data, and transmits the formatted data to the processor.

13. A non-transitory computer-readable storage medium comprising computer-executable instructions for assessing a brain injury of a patient, the computer-executable instructions comprising instructions for:
receiving brain wave test results, pupillary response test results, and eye tracking test results;
generating a first score based on a comparison of the brain wave test results to brain wave data stored in a normative database of reference results;
generating a second score based on a comparison of the pupillary response test results to pupillary data stored in the normative database of reference results;
generating a third score based on a comparison of eye tracking test results to eye tracking data stored in the normative database of reference results; and
generating a brain injury score based on the first score, the second score, and the third score.
